(19) United States
(12) Patent Application Publication
(10) Pub. No.: US 2016/0024108 A1
(43) Pub. Date: Jan. 28, 2016

(54) USE OF DITHIINE-TETRACARBOXIMIDES FOR CONTROLLING BACTERIAL HARMFUL ORGANISMS IN USEFUL PLANTS

(71) Applicant: BAYER CROPSCIENCE AKTIENGESELLSCHAFT, Monheim am Rhein (DE)

(72) Inventors: Thomas SEITZ, Langenfeld (DE); Christoph Andreas BRAUN, Duesseldorf (DE)

(21) Appl. No.: 14/772,624

(22) PCT Filed: Mar. 7, 2014
(86) PCT No.: PCT/EP2014/054491

§ 371 Date: Sep. 3, 2015

(30) Foreign Application Priority Data
Mar. 12, 2013 (EP) 13158696.8

(51) Int. Cl. C07D 495/14; A01N 43/90

(52) U.S. Cl. CPC C07D 495/14; A01N 43/90

(57) ABSTRACT

The present invention relates to the use of dithiine-tetracarboximides of formula (I) for controlling selected bacterial harmful organisms in useful plants, wherein the bacterial harmful organisms are selected from the group consisting of Acidovorax avenae, Burkholderia spec., Burkholderia glumae, Candidatus Liberibacter spec., Candidatus Liberibacter asiaticus, Corynebacterium, Erwinia spec. (Dickeya, Pectobacterium carotovorum, Erwinia amylovora), Pseudomonas syringae, Pseudomonas syringae pv. actinidae, Pseudomonas syringae pv. glycinea, Pseudomonas syringae pv. tomato, Pseudomonas syringae pv. lachrymans, Pseudomonas tumefaciens (=Agrobacterium tumefaciens), Streptomyces spp., Xanthomonas spp., Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. Viticola, Xanthomonas fragariae and Xanthomonas transluscens or Xylella fastidiosa. The present invention also relates to a method for controlling the selected bacterial harmful organisms in useful plants by treatment with a dithiine-tetracarboximides of formula (I).

The present invention relates to the use of dithiine-tetracarboximides of formula (I) for controlling selected bacterial harmful organisms in useful plants, wherein the bacterial harmful organisms are selected from the group consisting of Acidovorax avenae, Burkholderia spec., Burkholderia glumae, Candidatus Liberibacter specCandidatus Liberibacter asiaticus, Corynebacterium, Erwinia spec. (Dickeya, Pectobacterium carotovorum, Erwinia amylovora), Pseudomonas syringae, Pseudomonas syringae pv. actinidae, Pseudomonas syringae pv. glycinea, Pseudomonas syringae pv. tomato, Pseudomonas syringae pv. lachrymans, Pseudomonas tumefaciens (=Agrobacterium tumefaciens), Streptomyces spp., Xanthomonas spp., Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. viticola, Xanthomonas fragariae and Xanthomonas transluscens or Xylella fastidiosa. The present invention also relates to a method for controlling the selected bacterial harmful organisms in useful plants by treatment with a dithiine-tetracarboximides of formula (I).

### INTRODUCTION AND PRIOR ART

International patent application WO 2010/089055 A2 and the corresponding European patent application EP 2393363 A2 generally disclose the use of sulphur-containing heteroaromatic acid analogues according to a general formula (I) for controlling bacterial harmful organisms in useful plants.

Bacteria as pathogens in useful plants are encountered inter alia in temperate or warm and humid climates, where they cause bacterioses in a large number of useful plants with in some cases considerable economic losses.

Rice, for example, may be infected with Acidovorax avenae or Burkholderia glumae, causing brown stripe or bacterial grain rot, respectively.

Citrus greening disease (Huanglongbing, HLB, citrus vein phloem degeneration (CVPD), yellow shoot disease, leaf mottle yellow (in the Philippines), libukin (in Taiwan) and citrus dieback (in India)), caused by Candidatus Liberibacter spp., is probably the most deleterious disease of citrus and greatly reduces production, destroys the economic value of fruit and can ultimately lead to the death of the entire plant. Candidatus Liberibacter spp. is a genus of gram-negative bacteria in the Rhizobiaceae family. Members of the genus are plant pathogens, which are mostly transmitted by psyllids. The disease is distinguished by the common symptoms of yellowing of the veins and adjacent tissues; followed by yellowing or mottling of the entire leaf; followed by premature defoliation, dieback of twigs, decay of feeder rootlets and lateral roots, and decline in vigor; and followed by, ultimately, the death of the entire plant. Affected trees have stunted growth, bear multiple off-season flowers (most of which fall off), and produce small, irregularly-shaped fruit with a thick, pale peel that remains green at the bottom. Fruit from these trees tastes bitter. Infected trees do not recover. The control of HLB is based on the preventive control of the vectors using systemic insecticides and contact insecticides. However, the efficacy and activity spectrum of these compounds are not always completely satisfactory. Newly infected trees show the first symptoms after a latency period of 6-12 months. In addition, it is essential to eradicate infected trees to prevent further uptake by psyllids and spreading of the disease. There is no cure for Huanglongbing and efforts to control the disease have been slow because infected citrus plants are difficult to maintain, regenerate, and study. Researchers at the Agricultural Research Service have used Huanglongbing-infected lemon trees to infect periwinkle plants in an effort to study the disease. Periwinkle plants are easily infected with the disease and respond well when experimentally treated with antibiotics. Researchers are testing the effect of penicillin G sodium and the biocide 2,2-dibromo-3-nitrilopropionamide as potential treatments for infected citrus plants based on the positive results that were observed when applied to infected periwinkle. HLB bacteria live and multiply exclusively in the phloem of citrus trees. Hitherto, there are however only few bactericides for the curative control of HLB, e.g. the international application WO 2011/029536 A2 refers to the use of cyclic ketoenols against Candidatus liberibacter spp.

Citrus canker is a disease affecting citrus species that is caused by the bacterium Xanthomonas axonopodis pv. citri (=Xanthomonas campestris pv. citri). Infection causes lesions on the leaves, stems, and fruit of citrus trees, including lime, oranges, and grapefruit. While not harmful to humans, canker significantly affects the vitality of citrus trees, causing leaves and fruit to drop prematurely; a fruit infected with canker is safe to eat but too unsightly to be sold. The impact is worsened because the presence of citrus canker in an area triggers immediate quarantine restrictions, disrupting the movement of fresh fruit. Citrus canker is believed to have originated in the area of Southeast Asia-India. It is now also present in Japan, South and Central Africa, the Middle East, Bangladesh, the Pacific Islands, some countries in South America, and Florida. Some areas of the world have eradicated citrus canker and others have ongoing eradication programs (citrus groves have been destroyed in attempts to eradicate the disease), but the disease remains endemic in most areas where it has appeared. Because of its rapid spread, high potential for damage and impact on export sales and domestic trade, citrus canker is a significant threat to all citrus-growing regions.

The kiwifruit industry is widely affected by Pseudomonas ssp. infections, e.g. infection with Pseudomonas syringae pv. actinidae (Psa) was first identified in New Zealand and in Japan and Italy, too, where it is extremely damaging on Gold kiwifruit. Presently, intensive research and testing of possible solutions against Psa damage in kiwi fruits are carried out.

Infection with Erwinia species, for example, may cause the death of entire fruit plantations such as apples or pears. Also known is bacterial soft rot in potatoes, tumour formation in plants caused by infection with agrobacteria and also a large number of necrotic diseases when cereals such as wheat or rice, vegetables or citrus fruit are infected by Xanthomonas species.

The standard treatment against bacterial harmful organisms comprises the use of antibiotics such as e.g. streptomycin, blasticidin S or kasugamycin, which is, in principle, the only effective way for controlling bacteria in useful plants. However, this approach is adopted only in rare cases since these antibiotics rely on the same mechanisms of action as antibiotics used in human and veterinary medicine, and there are therefore huge reservations against the use of antibiotics in plant protection. There are concerns that the formation of resistance is promoted; moreover, most antibiotics are expensive and can frequently only be obtained by employing biotechnological methods, inter alia. Another approach for controlling bacteria in plant aims at the use of copper oxychloride, which is disadvantageous because of the necessity of high doses to be applied in the standard treatment. Copper oxychloride is e.g. used in controlling Pseudomonas syringae for example in the protection of tomatoes. Further, copper oxychloride is discussed as being phytotoxic and its use is more and more restricted as it is known to accumulate in the soil. In addition, copper oxychloride formulations normally leave visible residues on leaves and fruits, which is not appreciated and accepted by consumers.

There is therefore a great need for specific effective methods for controlling bacterial diseases in useful plants, which methods furthermore require only small amounts of substance to be applied and, in addition, do not damage the plants or harm human or animal health.

It has now been found that dithiino-tetracarboximides of formula (I), are particularly suitable for controlling bacterial harmful organisms of the group comprising one of the following organisms, preferably consisting each of the following organsims: Acidovorax avenae, Burkholderia spec., Burkholderia glumae, Candidatus Liberibacter spec., Corynebacterium, Erwinia spec., Pseudomonas syringae, Pseudomonas syringae pv. actinidae, Pseudomonas syringae pv. glycinea, Pseudomonas syringae pv. tomato, Pseudomonas syringae pv. lachrymans, Pseudomonas tumefaciens (=Agrobacterium tumefaciens) Streptomyces spp., Xanthomonas spp., Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. viticola, Xanthomonas fragariae and Xanthomonas transluscens or Xylella fastidiosa in useful plants.

### Problem to be Solved

It was the object of the present invention to provide novel active compounds for controlling selected bacterial harmful organisms in useful plants.

### DESCRIPTION OF THE INVENTION

The problem underlying the present invention has been solved by identifying the beneficial effects of di-thiino-tetracarboximide of formula (I)

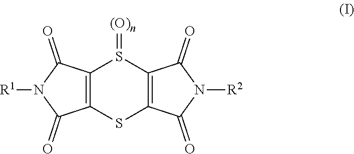

in which R1 and R2 are identical and represent methyl, ethyl, n-propyl or isopropyl, and n represents 0 or 1, or an agrochemically acceptable salt thereof,
in the treatment of useful plants against selected bacterial harmful organisms.

Preference is given to compounds of the formula (I) selected from the group consisting of
(I-1) 2,6-dimethyl-1H,5H-[1,4]dithiino[2,3-c:5,6-c′]dipyrrole-1,3,5,7(2H,6H)-tetrone (i.e. R1=R2=methyl, n=0)
(I-2) 2,6-diethyl-1H,5H-[1,4]dithiino[2,3-c:5,6-c′]dipyrrole-1,3,5,7(2H,6H)-tetrone (i.e. R1=R2=ethyl, n=0)
(I-3) 2,6-dipropyl-1H,5H-[1,4]dithiino[2,3-c:5,6-c′]dipyrrole-1,3,5,7(2H,6H)-tetrone (i.e. R1=R2=propyl, n=0)
(I-4) 2,6-diisopropyl-1H,5H-[1,4]dithiino[2,3-c:5,6-c′]dipyrrole-1,3,5,7(2H,6H)-tetrone (i.e. R1=R2=isopropyl, n=0)
(I-5) 2,6-dimethyl-1H,5H-[1,4]dithiino[2,3-c:5,6-c′]dipyrrole-1,3,5,7(2H,6H)-tetrone 4-oxide (i.e. R1=R2=methyl, n=1).

The dithiino-tetracarboximide of formula (I) according to the present invention are suitable in the use for controlling bacterial harmful organisms. According to the present invention bacterial harmful organisms include inter alia bacteria causing damage to plants or to a part of a plant.

Bacteria include inter alia Actinobacteria and Proteobacteria and are selected from the families of the Xanthomonadaceae, Pseudomonadaceae, Enterobacteriaceae, Microbacteriaceae, and Rhizobiaceae.

According to the present invention the bacterial harmful organisms are selected from the group selection comprising one of the following organisms, preferably consisting each of the following organisms: Acidovorax avenae (=Pseudomonas avenae, Pseudomonas avenae subsp. avenae, Pseudomonas rubrilineans), including e.g. Acidovorax avenae subsp. avenae (=Pseudomonas avenae subsp. avenae), Acidovorax avenae subsp. cattleyae (=Pseudomonas cattleyae), Acidovorax avenae subsp. citrulli (=Pseudomonas pseudoalcaligenes subsp. citrulli, Pseudomonas avenae subsp. citrulli));

Burkholderia spec., including e.g. Burkholderia andropogonis (=Pseudomonas andropogonis, Pseudomonas woodsii), Burkholderia caryophylli (=Pseudomonas caryophylli), Burkholderia cepacia (=Pseudomonas cepacia), Burkholderia gladioli (=Pseudomonas gladioli), Burkholderia gladioli pv. agaricicola (=Pseudomnas gladioli pv. agaricicola), Burkholderia gladioli pv. alliicola (=Pseusomonas gladioli pv. alliicola), Burkholderia gladioli pv. gladioli (=Pseudomonas gladioli, Pseudomonas gladioli pv. gladioli), Burkholderia glumae (=Pseudomonas glumae), Burkholderia plantarii (=Pseudomonas plantarii) Burkholderia solanacearum (=Ralstonia solanacearum);

Candidatus Liberibacter spec., including e.g. Liberibacter africanus (Laf), Liberibacter americanus (Lam), Liberibacter asiaticus (Las), Liberibacter europaeus (Leu), Liberibacter psyllaurous, Liberibacter solanacearum (Lso);

Corynebacterium, including e.g. Corynebacterium fascians, Corynebacterium flaccumfaciens pv. flaccumfaciens, Corynebacterium michiganensis, Corynebacterium michiganense pv. tritici, Corynebacterium michiganense pv. nebraskense, Corynebacterium sepedonicum;

Erwinia spec. including e.g. Erwinia amylovora, Erwinia ananas, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia dissolvers, Erwinia herbicola, Erwinia rhapontic, Erwinia stewartiii, Erwinia tracheiphila, Erwinia uredovora, Eriwina vitivora, Dickeya dedantii, Dickeya solani;

Pseudomonas syringae, including e.g. Pseudomonas syringae pv. actinidiae (Psa), Pseudomonas syringae pv. atrofaciens, Pseudomonas syringae pv. coronafaciens, Pseudomonas syringae pv. glycinea, Pseudomonas syringae pv. lachrymans, Pseudomonas syringae pv. maculicola Pseudomonas syringae pv. papulans, Pseudomonas syringae pv. striafaciens, Pseudomonas syringae pv. syringae, Pseudomonas syringae pv. tomato, Pseudomonas syringae pv. Tabaci, Pseudomonas tumefaciens (=Agrobacterium tumefaciens);

Streptomyces ssp., including e.g. Streptomyces acidiscabies, Streptomyces albidoflavus, Streptomyces candidus (=Actinomyces candidus), Streptomyces caviscabies, Streptomyces collinus, Streptomyces europaeiscabiei, Streptomyces intermedius, Streptomyces ipomoeae, Streptomyces luridiscabiei, Streptomyces niveiscabiei, Streptomyces puniciscabiei, Streptomyces retuculiscabiei, Streptomyces scabiei, Streptomyces scabies, Streptomyces setonii, Streptomyces steliiscabiei, Streptomyces turgidiscabies, Streptomyces wedmorensis;

Xanthomonas axonopodis, including e.g. Xanthomonas axonopodis pv. alfalfae (=Xanthomonas alfalfae), Xanthomonas axonopodis pv. aurantifolii (=Xanthomonas fuscans subsp. aurantifolii), Xanthomonas axonopodis pv. allii (=Xanthomonas campestris pv. allii), Xanthomonas axonopodis pv. axonopodis, Xanthomonas axonopodis pv. bauhiniae (=Xanthomonas campestris pv. bauhiniae), Xanthomonas axonopodis pv. begoniae (=Xanthomonas campestris pv. begoniae), Xanthomonas axonopodis pv. betlicola (=Xanthomonas campestris pv. betlicola), Xanthomonas axonopodis pv. biophyti (=Xanthomonas campestris pv. biophyti), Xanthomonas axonopodis pv. cajani (=Xanthomonas campestris pv. cajani), Xanthomonas axonopodis pv. cassavae (=Xanthomonas cassavae, Xanthomonas campestris pv. cassavae), Xanthomonas axonopodis pv. cassiae (=Xanthomonas campestris pv. cassiae), Xanthomonas axonopodis pv. citri (=Xanthomonas citri), Xanthomonas axonopodis pv. citrumelo (=Xanthomonas alfalfae subsp. citrumelonis), Xanthomonas axonopodis pv. clitoriae (=Xanthomonas campestris pv. clitoriae), Xanthomonas axonopodis pv. coracanae (=Xanthomonas campestris pv. coracanae), Xanthomonas axonopodis pv. cyamopsidis (=Xanthomonas campestris pv. cyamopsidis), Xanthomonas axonopodis pv. desmodii (=Xanthomonas campestris pv. desmodii), Xanthomonas axonopodis pv. desmodiigangetici (=Xanthomonas campestris pv. desmodiigangetici), Xanthomonas axonopodis pv. desmodiilaxiflori (=Xanthomonas campestris pv. desmodiilaxiflori), Xanthomonas axonopodis pv. desmodiirotundifolii (=Xanthomonas campestris pv. desmodiirotundifolii), Xanthomonas axonopodis pv. dieffenbachiae (=Xanthomonas campestris pv. dieffenbachiae), Xanthomonas axonopodis pv. erythrinae (=Xanthomonas campestris pv. erythrinae), Xanthomonas axonopodis pv. fascicularis (=Xanthomonas campestris pv. fasciculari), Xanthomonas axonopodis pv. glycines (=Xanthomonas campestris pv. glycines), Xanthomonas axonopodis pv. khayae (=Xanthomonas campestris pv. khayae), Xanthomonas axonopodis pv. lespedezae (=Xanthomonas campestris pv. lespedezae), Xanthomonas axonopodis pv. maculifoliigardeniae (=Xanthomonas campestris pv. maculifoliigardeniae), Xanthomonas axonopodis pv. malvacearum (=Xanthomonas citri subsp. malvacearum), Xanthomonas axonopodis pv. manihotis (=Xanthomonas campestris pv. manihotis), Xanthomonas axonopodis pv. martyniicola (=Xanthomonas campestris pv. martyniicola), Xanthomonas axonopodis pv. melhusii (=Xanthomonas campestris pv. melhusii), Xanthomonas axonopodis pv. nakataecorchori (=Xanthomonas campestris pv. nakataecorchori), Xanthomonas axonopodis pv. passiflorae (=Xanthomonas campestris pv. passiflorae), Xanthomonas axonopodis pv. patelii (=Xanthomonas campestris pv. patelii), Xanthomonas axonopodis pv. pedalii (=Xanthomonas campestris pv. pedalii), Xanthomonas axonopodis pv. phaseoli (=Xanthomonas campestris pv. phaseoli, Xanthomonas phaseoli), Xanthomonas axonopodis pv. phaseoli var. fuscans (=Xanthomonas fuscans), Xanthomonas axonopodis pv. phyllanthi (=Xanthomonas campestris pv. phyllanthi), Xanthomonas axonopodis pv. physalidicola (=Xanthomonas campestris pv. physalidicola), Xanthomonas axonopodis pv. poinsettiicola (=Xanthomonas campestris pv. poinsettiicola), Xanthomonas axonopodis pv. punicae (=Xanthomonas campestris pv. punicae), Xanthomonas axonopodis pv. rhynchosiae (=Xanthomonas campestris pv. rhynchosiae), Xanthomonas axonopodis pv. ricini (=Xanthomonas campestris pv. ricini), Xanthomonas axonopodis pv. sesbaniae (=Xanthomonas campestris pv. sesbaniae), Xanthomonas axonopodis pv. tamarindi (=Xanthomonas campestris pv. tamarindi), Xanthomonas axonopodis pv. vasculorum (=Xanthomonas campestris pv. vasculorum), Xanthomonas axonopodis pv. vesicatoria (=Xanthomonas campestris pv. vesicatoria, Xanthomonas vesicatoria), Xanthomonas axonopodis pv. vignaeradiatae (=Xanthomonas campestris pv. vignaeradiatae), Xanthomonas axonopodis pv. vignicola (=Xanthomonas campestris pv. vignicola), Xanthomonas axonopodis pv. vitians (=Xanthomonas campestris pv. vitians);

Xanthomonas campestris pv. musacearum, Xanthomonas campestris pv. pruni (=Xanthomonas arboricola pv. pruni), Xanthomonas fragariae; Xanthomonas campestris pv. viticola.

Xanthomonas translucens (=Xanthomonas campestris pv. hordei) including e.g. Xanthomonas translucens pv. arrhenatheri (=Xanthomonas campestris pv. arrhenatheri), Xanthomonas translucens pv. cerealis (=Xanthomonas campestris pv. cerealis), Xanthomonas translucens pv. graminis (=Xanthomonas campestris pv. graminis), Xanthomonas translucens pv. phlei (=Xanthomonas campestris pv. phlei), Xanthomonas translucens pv. phleipratensis (=Xanthomonas campestris pv. phleipratensis), Xanthomonas translucens pv. poae (=Xanthomonas campestris pv. poae), Xanthomonas translucens pv. secalis (=Xanthomonas campestris pv. secalis), Xanthomonas translucens pv. translucens (=Xanthomonas campestris pv. translucens), Xanthomonas translucens pv. undulosa (=Xanthomonas campestris pv. Undulosa);

Xanthomonas ampelina (=Xylophilus amelinus).

Xylella fastidiosa.

Preferably, the bacterial harmful organisms are selected from the group selection comprising one of the following organisms, preferably consisting each of the following organisms: Acidovorax avenae subsp. avenae (=Pseudomonas avenae subsp. avenae), Acidovorax avenae subsp. citrulli (=Pseudomonas pseudoalcaligenes subsp. citrulli, Pseudomonas avenae subsp. citrulli), Burkholderia glumae (=Pseudomonas glumae), Burkholderia solanacearum (=Ralstonia solanacearum), Candidatus Liberibacter spec. as defined above, Corynebacterium michiganense pv. nebraskense, Erwinia amylovora, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia herbicola, Erwinia stewartiii, Erwinia uredovora, Dickeya dedantii, Dickeya solani, Pseudomonas syringae, Pseudomonas syringae pv. actinidiae (Psa), Pseudomonas syringae pv. glycinea, Pseudomonas syringae pv. lachrymans, Pseudomonas syringae pv. papulans, Pseudomonas syringae pv. syringae, Pseudomonas syringae pv. tomato, Pseudomonas syringae pv. tabaci, Pseudomonas tumefaciens (=Agrobacterium tumefaciens), Streptomyces scabies, Xanthomonas ampelina, Xanthomonas axonopodis pv. citri, Xanthomonas axonopodis pv. glycines (=Xanthomonas campestris pv. glycines), Xanthomonas axonopodis pv. punicae (=Xanthomonas campestris pv. punicae), Xanthomonas axonopodis pv. vesicatoria (=Xanthomonas campestris pv. vesicatoria, Xanthomonas vesicatoria), Xanthomonas campestris, Xanthomonas campestris pv. musacearum, Xanthomonas campestris pv. pruni (=Xanthomonas arboricola pv. pruni), Xanthomonas campestris pv. viticola, Xanthomonas fragariae, Xanthomonas translucens pv. translucens (=Xanthomonas campestris pv. Translucens), Xylella fastidiosa.

In a more preferred aspect of the present invention the bacterial harmful organisms are selected from the group selection comprising one of the following organisms, preferably consisting each of the following organisms: Acidovorax avenae (=Pseudomonas avenae, Pseudomonas avenae subsp. avenae, Pseudomonas rubrilineans) as defined above, Burkholderia spec. as defined above, Burkholderia glumae, Candidatus Liberibacter spec. as defined above, Corynebacterium as defined above, Erwinia spec. as defined above, Erwinia amylovora, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia herbicola, Erwinia stewartiii, Erwinia uredovora, Dickeya dedantii, Dickeya solani, Pseudomonas syringae as defined above, Pseudomonas syringae pv. actinidae, Pseudomonas syringae pv. glycinea, Pseudomonas syringae pv. tomato, Pseudomonas syringae pv. lachrymans, Pseudomonas tumefaciens (=Agrobacterium tumefaciens), Streptomyces spp., Streptomyces scabies, Xanthomonas spp., Xanthomonas ampelina, Xanthomonas axonopodis as defined above, Xanthomonas axonopodis pv. citri, Xanthomonas axonopodis pv. glycines, Xanthomonas campestris, Xanthomonas campestris pv. musacearum, Xanthomonas campestris pv. pruni (=Xanthomonas arboricola pv. pruni), Xanthomonas campestris pv. viticola, Xanthomonas fragariae and Xanthomonas translucens (=Xanthomonas campestris pv. Hordei), Xylella fastidiosa as defined above.

Even more preferred is a selection comprising one of the following organisms, preferably consisting each of the following organisms:

Acidovorax avenae, Burkholderia spec., Burkholderia glumae, Candidatus Liberibacter spec., Liberibacter asiaticus (Las) Corynebacterium, Erwinia spec., Erwinia amylovora, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia herbicola, Erwinia stewartiii, Erwinia uredovora, Dickeya dedantii, Dickeya solani, Pseudomonas syringae, Pseudomonas syringae pv. actinidae, Pseudomonas syringae pv. glycinea, Pseudomonas syringae pv. tomato, Pseudomonas syringae pv. lachrymans, Pseudomonas tumefaciens (=Agrobacterium tumefaciens) Streptomyces spp., Xanthomonas spp., Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. viticola, Xanthomonas fragariae and Xanthomonas transluscens, or Xylella fastidiosa.

In an even more preferred aspect of the present invention the bacterial harmful organisms are selected from the group comprising one of the following organisms, preferably consisting each of the following organisms:

Acidovorax avenae, Burkholderia spec., Burkholderia glumae, Candidatus Liberibacter spec., Liberibacter asiaticus (Las), Corynebacterium, Erwinia amylovora, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia herbicola, Erwinia stewartiii, Erwinia uredovora, Dickeya dedantii, Dickeya solani, Pseudomonas syringae, Pseudomonas syringae pv. actinidae, Pseudomonas syringae pv. glycinea, Pseudomonas syringae pv. lachrymans, Pseudomonas syringae pv. tomato, Pseudomonas tumefaciens (=Agrobacterium tumefaciens), Streptomyces scabies, Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. viticola, Xanthomonas fragariae and Xanthomonas translucens or Xylella fastidiosa.

The most preferred selection comprising one of the following organisms, preferably consisting each of the following organisms:

Burkholderia glumae, Candidatus Liberibacter spec., Liberibacter asiaticus (Las), Xanthomonas ampelina, Xanthomonas axonopodis pv. citri, Erwinia amylovora, Erwinia carotovora (=Pectobacterium carotovorum), Dickeya dedantii, Dickeya solani, Pseudomonas syringae, Pseudomonas syringae pv. actinidae, Pseudomonas syringae pv. glycinea, Pseudomonas syringae pv. lachrymans, Pseudomonas syringae pv. tomato, Pseudomonas tumefaciens (=Agrobacterium tumefaciens), Xanthomonas axonopodis pv. glycines, Xanthomonas campestris pv. pruni, Xanthomonas campestris pv. viticola and Xanthomonas campestris or Xylella fastidiosa.

The dithiino-tetracarboximides of formula (I) according to the present invention can therefore be employed for protecting plants against attack by the abovementioned pathogens within a certain post-treatment period. The period within which protection is afforded generally extends from 1 to 10 days, preferably 1 to 7 days, after the treatment of the plants with the active compounds. Depending on the form of application, the accessibility of the active compounds to the plant can be controlled in a targeted manner

The good plant tolerance of the dithiino-tetracarboximides of formula (I) at the concentrations required for controlling plant diseases permits a treatment of aerial and subterranean plant parts, of vegetative propagation material, and of the soil.

The dithiino-tetracarboximides of formula (I) according to the present invention are also suitable for increasing the yield, show low toxicity and are well tolerated by plants.

In the context of the present invention, on application to plants an advantageous effect was observed.

In accordance with the invention, all plants may be treated. Plants are, in the present context, understood as meaning all plant parts and plant populations, such as desired and undesired wild plants or crop plants (including naturally occurring crop plants). Crop plants may be plants which can be obtained by traditional breeding and optimization methods or else by biotechnological and recombinant methods, or combinations of these methods, including the transgenic plants and including the plant varieties capable or not of being protected by Plant Breeders' Rights. Such methods are, for example, doubled haploids, protoplast fusion, random or targeted mutagenesis and also molecular or genetic markers.

Plant parts are intended to mean all aerial and subterranean parts and organs of the plants, such as herb, pseudostem, shoot, leaf, bract, leaf sheaths, petiole, lamina, flower and root, examples which may be mentioned being leaves, needles, stalks, stems, flowers, fruiting bodies, fruit, banana hand, bunches and seeds, and also roots, tubers, rhizomes, offshoots, suckers, secondary growth. The plant parts also include crop material and vegetative and generative propagation material, for example cuttings, tubers, rhizomes, slips and seeds.

As has already been mentioned above, all plants can be treated in accordance with the invention. In a preferred embodiment, plant species and plant varieties, and their parts, which are found in the wild or which are obtained by conventional biological breeding methods, such as hybridization, meristem cultures, micropropagation, somatic embryogenesis, direct organogenesis or protoplast fusion, are treated. In a further preferred embodiment, transgenic plants and plant varieties which have been obtained by recombinant methods, if appropriate in combination with traditional methods (genetically modified organisms), are treated, such as, for example, transformation by means of Agrobacterium or particle bombardment of embryogenic cells, and micropropagation. Plants include all plant parts as mentioned above.

It is especially preferred to treat, in accordance with the invention, plants of those plant varieties which are in each case commercially available or in use. Plant varieties are understood as meaning plants with new properties (“traits”) which have been obtained by conventional breeding, by mutagenesis or else by recombinant DNA techniques. They may be varieties, breeds, biotypes and genotypes.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNA interference [RNAi] technology). A heterologous gene that is located in the genome is also called a transgene. A transgene that is defined by its particular location in the plant genome is called a transformation or transgenic event.

Plants and plant varieties which are preferably to be treated according to the invention include all plants which have genetic material which imparts particularly advantageous, useful traits to these plants (whether obtained by breeding and/or biotechnological means).

Plants that may be treated according to the invention are hybrid plants that already express the characteristics of heterosis, or hybrid vigour, which results in generally higher yield, vigour, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants which can be treated in accordance with the invention and which may be mentioned are the following:

cotton, flax, grapevine, vegetables and fruits (for example kiwi, pineapple), such as Rosaceae sp. (for example pome fruits such as apples and pears, but also stone fruits such as apricots, cherries, almonds and peaches, and soft fruits such as strawberries), or grapevines such as Vitis sp. (for example Vitis vinifera, Vitis labrusca, Vitis riparia, Vitis rotundifolia, Vitis amurensis) or pomegranate from the genus of Punica, Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana plants and banana plantations as well as plantains), Rubiaceae sp. (for example coffee), Theaceae sp., Sterculiceae sp., Rutaceae sp. (for example citrus, lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers, melons, cucurbits, pumpkins), Alliaceae sp. (for example leeks, onions), Papilionaceae sp. (for example peas); major crop plants such as Gramineae sp. (for example corn, maize, turf, cereals such as wheat, rye, rice, barley, oats, sorghum, millet and triticale), Asteraceae sp. (for example sunflower), Brassicaceae sp. (for example cabbage such as white cabbage and red cabbage, broccoli, cauliflower, Brussels sprouts, pak choi, kohlrabi, small radishes, and also oilseed rape, mustard, horseradish and cress), Fabacae sp. (for example beans, peanuts), Papilionaceae sp. (for example soya beans), Solanaceae sp. (for example potatoes), Chenopodiaceae sp. (for example sugar beet, fodder beet, Swiss chard, beetroot); useful plants and ornamental plants in gardens and forests; and in each case genetically modified types of these plants.

Preferably, the dithiino-tetracarboximides of formula (I) of the present invention are used for the treatment in plants selected from the group comprising one of the following plants, preferably consisting each of the following plants:

vegetables and fruits (for example kiwi, melon, pineapple), such as Rosaceae sp. (for example pome fruits such as apples and pears, but also stone fruits such as apricots, cherries, almonds and peaches, and soft fruits such as strawberries), or grapevines from the genus of Vitis sp. (for example Vitis vinifera, Vitis labrusca, Vitis riparia, Vitis rotundifolia, Vitis amurensis) or pomegranate from the genus of Punica, Musaceae sp. (for example banana plants and banana plantations as well as plantains), Rutaceae sp. (for example citrus, lemons, oranges and grapefruit); vegetables, such as Solanaceae sp. (for example tomatoes), Cucurbitaceae sp. (for example cucumbers, melons, cucurbits, pumpkins), major crop plants such as Gramineae sp. (for example corn, maize, turf, cereals such as wheat, rye, rice, barley, oats, sorghum, millet and triticale), Brassicaceae sp. (for example cabbage such as white cabbage and red cabbage, broccoli, cauliflower, Brussels sprouts, pak choi, kohlrabi, small radishes, and also oilseed rape, mustard, horseradish and cress), Papilionaceae sp. (for example soya beans), Solanaceae sp. (for example potatoes); and in each case genetically modified types of these plants.

Even more preferred is the treatment of plants selected from the group comprising one of the following plants, preferably consisting each of the following plants:

fruits, vegetables, potatoes, cereals, corn, rice and soybeans.

Therefrom a further preferred selection relates to the group comprising one of the following plants, preferably consisting each of the following plants: kiwi, melon, grapewines, pineapple, pome fruits such as apples, pears and pomegranate, stone fruits such as peaches, soft fruits such as strawberries, banana plants and banana plantations as well as plantains, citrus, lemons, oranges and grapefruit; tomatoes, cucumbers, melons, cucurbits, corn, cereals such as wheat, rice, cabbage, cauliflower, soya beans, potatoes; and in each case genetically modified types of these plants.

The most preferred selection of useful plants to be treated in accordance with the present invention relates to: apples, bananas, grapewines, citrus, kiwi, melons, peaches, pears, pineapple, pome fruit, pomegranate, cabbage, cauliflower, cucumbers, cucurbits, tomatoes, potatoes, wheat, rice and soybeans.

And further to: citrus, kiwi, peaches, cucumbers, tomatoes, potatoes, wheat and soybeans.

A further preferred aspect of the present invention relates to the use of dithiino-tetracarboximides of formula (I) for controlling at least one of:

Acidovorax avenae and/or Burkholderia glumae in rice; Candidatus Liberibacter spec. and/or Xanthomonas axonopodis pv. citri in citrus; Corynebacterium in corn; Pseudomonas syringae pv. actinidae in Kiwi; Xanthomonas campestris in peaches, bananas and/or plantains; Xanthomonas axonopodis in pomegranate; Pseudomonas syringae pv. glycinea and/or Xanthomonas axonopodis in soybeans; Burkholderia spec. and/or Xanthomonas transluscens in cereals (preferably in wheat); Pseudomonas syringae, Pseudomonas syringae pv. tomato and/or Xanthomonas campestris in tomatoes; Pseudomonas syringae and/or Pseudomonas syringae pv. lachrymans in cucumbers; Erwinia carotovora, Erwinia carotovora subsp. atroseptica and/or Streptomyces scabies in potatoes; Erwinia carotovora in bananas and/or plantains; Xanthomonas ampelina, Xanthomonas campestris pv. viticola, Agrobacterium tumefaciens and Xylella fastidiosa in grapewines; Erwinia amylovora in apple and pears; Pectobacterium carotovorum in potatoes

Therein it is more preferred to use the dithiino-tetracarboximides of formula (I) for controlling at least one of: Acidovorax avenae and/or Burkholderia spec. (preferably Burkholderia glumae) in rice; Candidatus Liberibacter spec. and/or Xanthomonas axonopodis (preferably Xanthomonas axonopodis pv. citri) in citrus; Pseudomonas syringae (preferably Pseudomonas syringae pv. syringae pv. actinidae) in Kiwi; Xanthomonas campestris and/or Xanthomonas campestris pv. pruni in peaches; Pseudomonas syringae (preferably Pseudomonas syringae pv. glycinea) and/or Xanthomonas axonopodis (preferably Xanthomonas axonopodis pv. glycines (=Xanthomonas campestris pv. glycines) in soybeans; Burkholderia spec. and/or Xanthomonas transluscens in cereals; Pseudomonas syringae (preferably Pseudomonas syringae pv. tomato) and/or Xanthomonas campestris in tomatoes; Pseudomonas syringae and/or Pseudomonas syringae pv. lachrymans in cucumbers; as well as Erwinia atroseptica, Erwinia carotovora and/or Streptomyces scabies in potatoes; Xanthomonas ampelina, Xanthomonas campestris pv. viticola, Agrobacterium tumefaciens and Xylella fastidiosa in grapewines; Erwinia amylovora in apple and pears; Pectobacterium carotovorum in potatoes.

Most preferred is to use the dithiino-tetracarboximides of formula (I) for controlling Burkholderia glumae in rice, Liberibacter spec. and/or Xanthomonas axonopodis pv. citri in citrus, Pseudomonas syringae pv. actinidiae (Psa) in kiwi, Pseudomonas syringae pv. glycinea and/or Xanthomonas axonopodis pv. glycines in soybeans, Pseudomonas syringae and/or Pseudomonas syringae pv. tomato in tomato and Xanthomonas campestris and/or Xanthomonas campestris pv. pruni in peaches, Pseudomonas syringae and/or Pseudomonas syringae pv. lachrymans in cucumbers, Xanthomonas ampelina, Xanthomonas campestris pv. viticola, Agrobacterium tumefaciens and Xylella fastidiosa in grapewines; Erwinia amylovora in apple and pears; Pectobacterium carotovorum in potatoes.

### Application Forms

The treatment according to the invention of the plants and plant parts with the active compound combinations or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by incrusting, by coating with one or more coats, etc. Preference is given to application by dipping, spraying, atomizing, irrigating, evaporating, dusting, fogging, broadcasting, foaming, painting, spreading-on, watering (drenching) and drip irrigating. Also encompassed by the present invention is nursery box treatment.

In an especially preferred embodiment of the present invention, dithiino-tetracarboximides of formula (I) or their formulations are used for application in the form of solutions, emulsions or suspensions to be applied by spraying, for the treatment of vegetative propagation material, or for rhizome or foliar application.

Depending on its respective physical and/or chemical properties, the selected dithiino-tetracarboximide of formula (I) can be converted into the customary formulations, such as solutions, emulsions, suspensions, powders, foams, pastes, granules, sachets, aerosols, microencapsulations in polymeric substances, and ULV cold- and hot-fogging formulations.

These formulations are prepared in a known manner, for example by mixing the dithiino-tetracarboximides of formula (I) with extenders, that is to say liquid solvents, pressurized liquefied gases and/or solid carriers, optionally with the use of surfactants, that is emulsifiers and/or dispersants and/or foam formers. If water is used as the extender, it is possible for example also to use organic solvents as cosolvents. Liquid solvents which are suitable in the main are: aromatics such as xylene, toluene or alkyl [symbol] naphthalenes, chlorinated aromatics or chlorinated aliphatic hydrocarbons such as chlorobenzenes, chloroethylenes or methylene chloride, aliphatic hydrocarbons, such as cyclohexane or paraffins, for example mineral oil fractions, alcohols such as butanol or glycol, and their ethers and esters, ketones such as acetone, methyl ethyl ketone, methyl isobutyl ketone or cyclohexanone, strongly polar solvents such as dimethylformamide and di [symbol] ime [symbol] ithyl sulph [symbol] oxide, and water, and also mineral, animal and vegetable oils such as, for example, palm oil or other plant seed oils. Liquefied gaseous extenders or carriers are understood as meaning those liquids which are gaseous at normal temperature and under normal pressure, for example aerosol propellants such as halohydrocarbons and butane, propane, nitrogen and carbon dioxide.

Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, pumice, marble, sepiolite, dolomite, and synthetic granules of inorganic and organic meals, and granules of organic material such as sawdust, coconut shells, maize cobs and tobacco stalks. Emulsifiers and/or foam formers which are suitable are: for example nonionic, cationic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, and protein hydrolysates. Suitable dispersants are: for example, lignosulphite waste liquors and methylcellulose.

Adhesives such as carboxymethylcellulose, natural and synthetic polymers in the form of powders, granules or latices, such as gum arabic, polyvinyl alcohol, polyvinyl acetate, and natural phospholipids such as cephalins and lecithins, and synthetic phospholipids, may be used in the formulations. Further additives may be mineral and vegetable oils.

It is possible to use colorants such as inorganic pigments, for example iron oxide, titanium oxide, Prussian Blue, and organic dyestuffs, such as alizarin, azo and metal phthalocyanine dyestuffs, and trace nutrients, such as salts of iron, manganese, boron, copper, cobalt, molybdenum and zinc.

In general, the formulations contain between 0.1 and 95% by weight of active compound (dithiino-tetracarboximide of formula (I)), preferably between 0.5 and 90%.

The control of the selected bacterial harmful organisms by treating the vegetative propagation material of plants has been known for a long time and is the subject of continuous improvements. However, the treatment of vegetative propagation material involves a series of problems which cannot always be solved in a satisfactory manner. Thus, it is desirable to develop methods for protecting the vegetative propagation material and the germinating plant which do away with, or at least markedly reduce, the additional application of plant protection products after planting or after emergence of the plants. It is furthermore desirable to optimize the amount of the active compound employed such that the vegetative propagation material and the germinating plant are protected the best possible from attack by the bacterial harmful organisms without, however, damaging the plant itself by the active compound employed. In particular, methods for the treatment of vegetative propagation material should also take into consideration the intrinsic properties of transgenic plants in order to achieve an optimal protection of the vegetative propagation material and the germinating plant while keeping the application rate of plant protection products as low as possible.

The present invention therefore relates in particular also to a method of protecting vegetative propagation material and germinating plants from attack by the selected bacterial harmful organisms, by treating the seed and the vegetative propagation material with a compound or formulation according to the invention.

The invention also relates to the use of the compounds according to the invention for the treatment of vegetative propagation material for protecting the vegetative propagation material and the germinating plant from the selected bacterial harmful organisms.

One of the advantages of the present invention is that, owing to the special systemic properties of the compounds according to the invention, the treatment of the vegetative propagation material with these compounds protects not only the vegetative propagation material itself, but also the plants which it gives rise to after planting, from the bacterial harmful organisms. In this manner, the immediate treatment of the crop at the time of planting, or shortly thereafter, can be dispensed with.

Another advantage is that the compounds according to the invention can be employed in particular also in transgenic vegetative propagation material.

The compounds according to the invention are suitable for protecting vegetative propagation material of any plant variety which is employed in agriculture, in the greenhouse, in forests or in horticulture. In particular, this is vegetative propagation material of the plants as defined and preferred herein.

Within the scope of the present invention, the compounds according to the invention are applied to the vegetative propagation material either alone or in a suitable formulation. Preferably, the vegetative propagation material is treated in a state in which it is sufficiently stable such that no damage occurs during the treatment. In general, the vegetative propagation material can be treated at any point in time between harvesting and planting out. Usually, vegetative propagation material is used which has been separated from the plant and freed from cobs, shells, stalks, coats, hairs or fruit flesh.

When treating the vegetative propagation material, care must be taken in general that the amount of the compound or formulation according to the invention, and/or of further additives, applied to the vegetative propagation material is chosen such that the germination of the vegetative propagation material is not adversely affected, or that the plant which it gives rise to is not damaged. This must be considered in particular in the case of active compounds which, at certain application rates, may have phytotoxic effects.

The compounds or formulations according to the invention can be applied directly, that is to say without containing further components and without having been diluted. In general, it is preferred to apply the compounds or formulations to the vegetative propagation material in the form of a suitable formulation. Suitable formulations and methods for the treatment of seed and of vegetative propagation material are known to the skilled worker.

The compounds or formulations which can be used in accordance with the invention can be converted into the customary formulations, such as solutions, emulsions, suspensions, powders, foams and ULV formulations.

These formulations are prepared in the known manner by mixing the dithiino-tetracarboximides of formula (I) with customary additives, such as, for example, customary extenders and also solvents or diluents, colorants, wetters, dispersants, emulsifiers, antifoams, preservatives, secondary thickeners, adhesives, gibberellins, mineral and vegetable oils, and also water.

Colorants which may be present in the formulations which can be used in accordance with the invention are all colorants which are customary for such purposes. In this context, both pigments, which are sparingly soluble in water, and dyes, which are soluble in water, may be used. Examples which may be mentioned are the colorants known by the names Rhodamin B, C.I. Pigment Red 112 and C.I. Solvent Red 1.

Wetters which may be present in the formulations which can be used in accordance with the invention are all substances which are customary for formulating agrochemical active compounds and which promote wetting. Alkylnaphthalenesulphonates, such as diisopropyl- or diisobutylnaphtha [symbol] lenesulphonates, may preferably be used.

Suitable dispersants and/or emulsifiers which may be present in the formulations which can be used in accordance with the invention are all nonionic, anionic and cationic dispersants which are conventionally used for the formulation of agrochemical active compounds. The following may be used by preference: nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristyrylphenol polyglycol ethers and their phosphated or sulphated derivatives. Suitable anionic dispersants are, in particular, lignosulphonates, salts of polyacrylic acid, and arylsulphonate/formaldehyde condensates.

Antifoams which may be present in the formulations which can be used in accordance with the invention are all foam-inhibitor substances which are conventionally used for the formulation of agrochemical active compounds. Silicone antifoams and magnesium stearate may be used by preference.

Preservatives which may be present in the formulations which can be used in accordance with the invention are all substances which can be employed for such purposes in agrochemical compositions. Examples which may be mentioned are dichlorophene and benzyl alcohol hemiformal.

Secondary thickeners which may be present in the formulations which can be used in accordance with the invention are all substances which can be employed for such purposes in agrochemical compositions. Cellulose derivatives, acrylic acid derivatives, xanthan, modified clays and highly disperse silica are preferably suitable.

Adhesives which may be present in the formulations which can be used in accordance with the invention are all customary binders which can be used in mordants. Polyvinylpyrrolidone, polyvinyl acetate, polyvinyl alcohol and tylose may be mentioned by preference.

Gibberellins which may be present in the formulations which can be used in accordance with the invention are preferably Gibberellin A1, Gibberellin A3 (gibberellic acid), Gibberellin A4, Gibberellin A7. Especially preferred is gibberellic acid.

The gibberellins are known (cf. R. Wegler “Chemie der Pflanzenschutz- and Schädlingsbekämpfungsmittel” [Chemistry of plant protection and pesticide agents], volume 2, Springer Verlag, Berlin-Heidelberg-New York, 1970, pages 401-412).

The formulations which can be used in accordance with the invention can be employed, for the treatment of various types of seed, either directly or after previously having been diluted with water. Thus, the concentrates or the preparations obtainable therefrom by dilution with water can be employed for dressing the seed. The formulations which can be used in accordance with the invention, or their diluted preparations, can also be employed for treating the vegetative propagation material of transgenic plants. Here, additional synergistic effects may also occur in combination with the substances formed by expression.

The application rate of the formulations which can be used in accordance with the invention can be varied within a substantial range. It depends on the respective active compound content in the formulations, and on the vegetative propagation material. As a rule, the application rates of active compound are between 0.001 and 50 g per kilogram of vegetative propagation material, preferably between 0.01 and 15 g per kilogram of vegetative propagation material.

### Combinations/Formulations

The preferred dithiino-tetracarboximides of formula (I) of the present invention (I-1), (I-2), (I-3), (I-4) and (I-5) can be employed as such or, in formulations, also in a combination with known bactericides, fungicides, acaricides, nematicides, herbicides, insecticides, micronutrients and micronutrient-containing compounds, safeners, lipochito-oligosaccharide compounds (LCO), soil-improvement products or products for reducing plant stress, for example Myconate, in order to widen the spectrum of action or to prevent the development of resistance, for example.

In the meaning of the invention, a lipochito-oligosaccharide (LCO) compound is a compound having the general LCO structure, i.e. an oligomeric backbone of β-1,4-linked N-acetyl-D-glucosamine residues with a N-linked fatty acyl chain at the non-reducing end, as described in U.S. Pat. No. 5,549,718; U.S. Pat. No. 5,646,018; U.S. Pat. No. 5,175,149; and U.S. Pat. No. 5,321,011. This basic structure may contain modifications or substitutions found in naturally occurring LCO's, such as those described in Spaink, Critical Reviews in Plant Sciences 54: 257-288, 2000; D'Haeze and Holsters, Glycobiology 12: 79R-105R, 2002. Naturally occurring LCO's are defined as compounds which can be found in nature. This basic structure may also contain modifications or substitutions which have not been found so far in naturally occurring LCO's. Examples of such analogs for which the conjugated amide bond is mimicked by a benzamide bond or which contain a function of benzylamine type are the following compounds of formula (I) which are described in WO 2005/063784 and WO 2008/071672, the content of which is incorporated herein by reference. The LCO's compounds may be isolated directly from a particular culture of Rhizobiaceae bacterial strains, synthesized chemically, or obtained chemo-enzymatically. Via the latter method, the oligosaccharide skeleton may be formed by culturing of recombinant bacterial strains, such as Escherichia coli, in a fermenter, and the lipid chain may then be attached chemically. LCO's used in embodiments of the invention may be recovered from natural Rhizobiaceae bacterial strains that produce LCO's, such as strains of Azorhizobium, Bradyrhizobium (including B. japonicum), Mesorhizobium, Rhizobium (including R. leguminosarum), Sinorhizobium (including S. meliloti), or from bacterial strains genetically engineered to produce LCO's. These methods are known in the art and have been described, for example, in U.S. Pat. Nos. 5,549,718 and 5,646,018, which are incorporated herein by reference. Hungria and Stacey (Soil Biol. Biochem. 29: 819-830, 1997) list specific LCO structures that are produced by different rhizobial species. LCO's may be utilized in various forms of purity and may be used alone or with rhizobia. Methods to provide only LCO's include simply removing the rhizobial cells from a mixture of LCOs and rhizobia, or continuing to isolate and purify the LCO molecules through LCO solvent phase separation followed by HPLC chromatography as described by Lerouge, et. al (U.S. Pat. No. 5,549,718). Purification can be enhanced by repeated HPLC, and the purified LCO molecules can be freeze-dried for long-term storage. This method is acceptable for the production of LCO's from all genera and species of the Rhizobiaceae. Commercial products containing LCO's are available, such as OPTIMIZE® (EMD Crop BioScience). LCO compounds, which can be identical or not to naturally occurring LCO's, may also be obtained by chemical synthesis and/or through genetic engineering. Synthesis of precursor oligosaccharide molecules for the construction of LCO by genetically engineered organisms is disclosed in Samain et al., Carbohydrate Research 302: 35-42, 1997. Preparation of numerous LCOs compounds wherein the oligosaccharide skeleton is obtained by culturing recombinant bacterial strains, such as recombinant Escherichia coli cells harboring heterologous gene from rhizobia, and wherein the lipid chain is chemically attached is disclosed in WO 2005/063784 and WO 2008/07167, the content of which is incorporated herein by reference. Examples of lipochito-oligosaccharide compounds include, but are not limited to LCO compounds specifically disclosed in WO 2010/125065.

Preferably the dithiino-tetracarboximides of formula (I) are present in a composition comprising at least one further compound selected from the group consisting of bactericides, antibiotics, fungicides, insecticides, herbicides, micronutrients and micronutrient-containing compounds, and lipochito-oligosaccharide compounds (LCO). Preferably, this at least one further compound is selected from the group consisting of:

Antibiotics such as kasugamycin, streptomycin, oxytetracyclin, validamycin, gentamycin, aureofungin, blasticidin-S, cycloheximide, griseofulvin, moroxydine, natamycin, polyoxins, polyoxorim and combinations thereof.

### Fungicides:

(1) Inhibitors of the ergosterol biosynthesis, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-[1-(4-methoxyphenoxy)-3,3-dimethylbutan-2-yl]1H-imidazole-1-carbothioate.

(2) inhibitors of the respiratory chain at complex I or II, for example bixafen, boscalid, carboxin, diflumetorim, fenfuram, fluopyram, flutolanil, fluxapyroxad, furametpyr, furmecyclox, isopyrazam (mixture of syn-epimeric racemate 1RS,4SR,9RS and anti-epimeric racemate 1RS,4SR,9SR), isopyrazam (anti-epimeric racemate 1RS,4SR,9SR), isopyrazam (anti-epimeric enantiomer 1R,4S,9S), isopyrazam (anti-epimeric enantiomer 1S,4R,9R), isopyrazam (syn epimeric racemate 1RS,4SR,9RS), isopyrazam (syn-epimeric enantiomer 1R,4S,9R), isopyrazam (syn-epimeric enantiomer 1S,4R,9S), mepronil, oxycarboxin, penflufen, penthiopyrad, sedaxane, thifluzamide, 1-methyl-N-[2-(1,1,2,2-tetrafluoroethoxy)phenyl]-3-(trifluoromethyl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, 5,8-difluoro-N-[2-(2-fluoro-4-{[4-(trifluoromethyl)pyridin-2-yl]oxy}phenyl)ethyl]quinazolin-4-amine, N-[9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.

(3) inhibitors of the respiratory chain at complex III, for example ametoctradin, amisulbrom, azoxystrobin, cyazofamid, coumethoxystrobin, coumoxystrobin, dimoxystrobin, enestroburin, famoxadone, fenamidone, fenoxystrobin, fluoxastrobin, kresoxim-methyl, metominostrobin, orysastrobin, picoxystrobin, pyraclostrobin, pyrametostrobin, pyraoxystrobin, pyribencarb, triclopyricarb, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, methyl(2E)-2-{2-[({cyclopropyl[(4-methoxyphenyl)imino]methyl}sulfanyl)methyl]phenyl}-3-methoxyprop-2-enoate, N-(3-ethyl-3,5,5-trimethylcyclohexyl)-3-(formylamino)-2-hydroxybenzamide, 2-{2-[(2,5-dimethylphenoxy)methyl]phenyl}-2-methoxy-N-methylacetamide and (2R)-2-{2-[(2,5-dimethylphenoxy)methyl]phenyl}-2-methoxy-N-methylacetamide.

(4) Inhibitors of the mitosis and cell division, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)[1,2,4]triazolo[1,5-a]pyrimidine and 3-chloro-5-(6-chloropyridin-3-yl)-6-methyl-4-(2,4,6-trifluorophenyl)pyridazine.

(5) Compounds capable to have a multisite action, for example bordeaux mixture, captafol, captan, chlorothalonil, copper hydroxide, copper naphthenate, copper oxide, copper oxychloride, copper(2+) sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulphur and sulphur preparations including calcium polysulphide, thiram, tolylfluanid, zineb and ziram.

(6) Compounds capable to induce a host defence, for example acibenzolar-S-methyl, isotianil, probenazole and tiadinil.

(7) Inhibitors of the amino acid and/or protein biosynthesis, for example andoprim, blasticidin-S, cyprodinil, kasugamycin, kasugamycin hydrochloride hydrate, mepanipyrim, pyrimethanil and 3-(5-fluoro-3,3,4,4-tetramethyl-3,4-dihydroisoquinolin-1-yl)quinoline.

(8) Inhibitors of the ATP production, for example fentin acetate, fentin chloride, fentin hydroxide and silthiofam.

(9) Inhibitors of the cell wall synthesis, for example benthiavalicarb, dimethomorph, flumorph, iprovalicarb, mandipropamid, polyoxins, polyoxorim, validamycin A and valifenalate.

(10) Inhibitors of the lipid and membrane synthesis, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.

(11) Inhibitors of the melanine biosynthesis, for example carpropamid, diclocymet, fenoxanil, phthalide, pyroquilon, tricyclazole and 2,2,2-trifluoroethyl {3-methyl-1-[(4-methylbenzoyl)amino]butan-2-yl}carbamate.

(12) Inhibitors of the nucleic acid synthesis, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.

(13) Inhibitors of the signal transduction, for example chlozolinate, fenpiclonil, fludioxonil, iprodione, procymidone, quinoxyfen and vinclozolin.

(14) Compounds capable to act as an uncoupler, for example binapacryl, dinocap, ferimzone, fluazinam and meptyldinocap.

(15) Further compounds, for example benthiazole, bethoxazin, capsimycin, carvone, chinomethionat, pyriofenone (chlazafenone), cufraneb, cyflufenamid, cymoxanil, cyprosulfamide, dazomet, debacarb, dichlorophen, diclomezine, difenzoquat, difenzoquat methylsulphate, diphenylamine, ecomate, fenpyrazamine, flumetover, fluoroimide, flusulfamide, flutianil, fosetyl-aluminium, fosetyl-calcium, fosetyl-sodium, hexachlorobenzene, irumamycin, methasulfocarb, methyl isothiocyanate, metrafenone, mildiomycin, natamycin, nickel dimethyldithiocarbamate, nitrothal-isopropyl, octhilinone, oxamocarb, oxyfenthiin, pentachlorophenol and salts, phenothrin, phosphorous acid and its salts, propamocarb-fosetylate, propanosine-sodium, proquinazid, pyrimorph, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one, pyrrolnitrine, tebufloquin, tecloftalam, tolnifanide, triazoxide, trichlamide, zarilamid, (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]pyridine, 2-phenylphenol and salts, 3-(4,4,5-trifluoro-3,3-dimethyl-3,4-dihydroisoquinolin-1-yl)quinoline, 3,4,5-trichloropyridine-2,6-dicarbonitrile, 3-[5-(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]pyrimidin-4-amine, 5-fluoro-2-[(4-methylbenzyl)oxy]pyrimidin-4-amine, 5-methyl-6-octyl[1,2,4]triazolo[1,5-a]pyrimidin-7-amine, ethyl(2Z)-3-amino-2-cyano-3-phenylprop-2-enoate, N′-(4-{[3-(4-chlorobenzyl)-1,2,4-thiadiazol-5-yl]oxy}-2,5-dimethylphenyl)-N-ethyl-N-methylimidoformamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(E)-[(cyclopropylmethoxy)imino][6-(difluoromethoxy)-2,3-difluorophenyl]methyl}-2-phenylacetamide, N-{(Z)-[(cyclopropylmethoxy)imino][6-(difluoromethoxy)-2,3-difluorophenyl]methyl}-2-phenylacetamide, N′-{4-[(3-tert-butyl-4-cyano-1,2-thiazol-5-yl)oxy]-2-chloro-5-methylphenyl}-N-ethyl-N-methylimidoformamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, pentyl {6-[({[(1-methyl-1H-tetrazol-5-yl)(phenyl)methylidene]amino}oxy)methyl]pyridin-2-yl}carbamate, phenazine-1-carboxylic acid, quinolin-8-ol, quinolin-8-ol sulfate (2:1) and tert-butyl {6-[({[(1-methyl-1H-tetrazol-5-yl)(phenyl)methylene]amino}oxy)methyl]pyridin-2-yl}carbamate.

(16) Further compounds, for example 1-methyl-3-(trifluoromethyl)-N-[2′-(trifluoromethyl)biphenyl-2-yl]-1H-pyrazole-4-carboxamide, N-(4′-chlorobiphenyl-2-yl)-3-(difluoromethyl)-1-methyl-1H-pyrazole-4-carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1-yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N-(4′-ethynylbiphenyl-2-yl)-5-fluoro-1,3-dimethyl-1H-pyrazole-4-carboxamide, 2-chloro-N-(4′-ethynylbiphenyl-2-yl)pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1H-pyrazole-4-carboxamide, 5-fluoro-N-[4′-(3-methoxy-3-methylbut-1-yn-1-yl)biphenyl-2-yl]-1,3-dimethyl-1H-pyrazole-4-carboxamide, 2-chloro-N-[4′-(3-methoxy-3-methylbut-1-yn-1-yl)biphenyl-2-yl]pyridine-3-carboxamide, (5-bromo-2-methoxy-4-methylpyridin-3-yl)(2,3,4-trimethoxy-6-methylphenyl)methanone, N-[2-(4-{[3-(4-chlorophenyl)prop-2-yn-1-yl]oxy}-3-methoxyphenyl)ethyl]-N2-(methylsulfonyl)valinamide, 4-oxo-4-[(2-phenylethyl)amino]butanoic acid and but-3-yn-1-yl {6-[({[(Z)-(1-methyl-1H-tetrazol-5-yl)(phenyl)methylene]amino}oxy)methyl]pyridin-2-yl}carbamate, and combinations thereof.

### Insecticides, Acaricides, and Nematicides:

(1) Acetylcholinesterase (AChE) inhibitors, for example carbamates, e.g. Alanycarb, Aldicarb, Bendiocarb, Benfuracarb, Butocarboxim, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiodicarb, Thiofanox, Triazamate, Trimethacarb, XMC, and Xylylcarb; or organophosphates, e.g. Acephate, Azamethiphos, Azinphos-ethyl, Azinphos-methyl, Cadusafos, Chlorethoxyfos, Chlorfenvinphos, Chlormephos, Chlorpyrifos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fosthiazate, Heptenophos, Imicyafos, Isofenphos, Isopropyl O-(methoxyaminothio-phosphoryl) salicylate, Isoxathion, Malathion, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-methyl, Profenofos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, and Vamidothion.

(2) GABA-gated chloride channel antagonists, for example cyclodiene organochlorines, e.g. Chlordane and Endosulfan; or phenylpyrazoles (fiproles), e.g. Ethiprole and Fipronil.

(3) Sodium channel modulators/voltage-dependent sodium channel blockers, for example pyrethroids, e.g. Acrinathrin, Allethrin, d-cis-trans Allethrin, d-trans Allethrin, Bifenthrin, Bioallethrin, Bioallethrin S-cyclopentenyl isomer, Bioresmethrin, Cycloprothrin, Cyfluthrin, beta-Cyfluthrin, Cyhalothrin, lambda-Cyhalothrin, gamma-Cyhalothrin, Cypermethrin, alpha-Cypermethrin, beta-Cypermethrin, theta-Cypermethrin, zeta-Cypermethrin, Cyphenothrin [(1R)-trans isomers], Deltamethrin, Empenthrin [(EZ)-(1R) isomers), Esfenvalerate, Etofenprox, Fenpropathrin, Fenvalerate, Flucythrinate, Flumethrin, tau-Fluvalinate, Halfenprox, Imiprothrin, Kadethrin, Permethrin, Phenothrin [(1R)-trans isomer), Prallethrin, Pyrethrine (pyrethrum), Resmethrin, Silafluofen, Tefluthrin, Tetramethrin, Tetramethrin [(1R) isomers)], Tralomethrin, and Transfluthrin; or DDT; or Methoxychlor.

(4) Nicotinic acetylcholine receptor (nAChR) agonists, for example neonicotinoids, e.g. Acetamiprid, Clothianidin, Dinotefuran, Imidacloprid, Nitenpyram, Thiacloprid, and Thiamethoxam; or Nicotine.

(5) Nicotinic acetylcholine receptor (nAChR) allosteric activators, for example spinosyns, e.g. Spinetoram and Spinosad.

(6) Chloride channel activators, for example avermectins/milbemycins, e.g. Abamectin, Emamectin benzoate, Lepimectin, and Milbemectin.

(7) Juvenile hormone mimics, for example juvenile hormon analogues, e.g. Hydroprene, Kinoprene, and Methoprene; or Fenoxycarb; or Pyriproxyfen.

(8) Miscellaneous non-specific (multi-site) inhibitors, for example alkyl halides, e.g. Methyl bromide and other alkyl halides; or Chloropicrin; or Sulfuryl fluoride; or Borax; or Tartar emetic.

(9) Selective homopteran feeding blockers, e.g. Pymetrozine; or Flonicamid.

(10) Mite growth inhibitors, e.g. Clofentezine, Hexythiazox, and Diflovidazin; or Etoxazole.

(11) Microbial disruptors of insect midgut membranes, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT crop proteins: Cry1Ab, Cry1Ac, Cry1Fa, Cry2Ab, mCry3A, Cry3Ab, Cry3Bb, Cry34/35Ab1.

(12) Inhibitors of mitochondrial ATP synthase, for example Diafenthiuron; or organotin miticides, e.g. Azocyclotin, Cyhexatin, and Fenbutatin oxide; or Propargite; or Tetradifon.

(13) Uncouplers of oxidative phoshorylation via disruption of the proton gradient, for example Chlorfenapyr, DNOC, and Sulfluramid.

(14) Nicotinic acetylcholine receptor (nAChR) channel blockers, for example Bensultap, Cartap hydrochloride, Thiocyclam, and Thiosultap-sodium.

(15) Inhibitors of chitin biosynthesis, type 0, for example Bistrifluron, Chlorfluazuron, Diflubenzuron, Flucycloxuron, Flufenoxuron, Hexaflumuron, Lufenuron, Novaluron, Noviflumuron, Teflubenzuron, and Triflumuron.

(16) Inhibitors of chitin biosynthesis, type 1, for example Buprofezin.

(17) Moulting disruptors, for example Cyromazine.

(18) Ecdysone receptor agonists, for example Chromafenozide, Halofenozide, Methoxyfenozide, and Tebufenozide.

(19) Octopamine receptor agonists, for example Amitraz.

(20) Mitochondrial complex III electron transport inhibitors, for example Hydramethylnon; or Acequinocyl; or Fluacrypyrim.

(21) Mitochondrial complex I electron transport inhibitors, for example METI acaricides, e.g. Fenazaquin, Fenpyroximate, Pyrimidifen, Pyridaben, Tebufenpyrad, and Tolfenpyrad; or Rotenone (Derris).

(22) Voltage-dependent sodium channel blockers, e.g. Indoxacarb; or Metaflumizone.

(23) Inhibitors of acetyl CoA carboxylase, for example tetronic and tetramic acid derivatives, e.g. Spirodiclofen, Spiromesifen, and Spirotetramat.

(24) Mitochondrial complex IV electron transport inhibitors, for example phosphines, e.g. Aluminium phosphide, Calcium phosphide, Phosphine, and Zinc phosphide; or Cyanide.

(25) Mitochondrial complex II electron transport inhibitors, for example Cyenopyrafen.

(28) Ryanodine receptor modulators, for example diamides, e.g. Chlorantraniliprole and Flubendiamide.

Further active ingredients with unknown or uncertain mode of action, for example Amidoflumet, Azadirachtin, Benclothiaz, Benzoximate, Bifenazate, Bromopropylate, Chinomethionat, Cryolite, Cyantraniliprole (Cyazypyr), Cyflumetofen, Dicofol, Diflovidazin, Fluensulfone, Flufenerim, Flufiprole, Fluopyram, Fufenozide, Imidaclothiz, Iprodione, Meperfluthrin, Pyridalyl, Pyrifluquinazon, Tetramethylfluthrin, and iodomethane; furthermore products based on Bacillus firmus (including but not limited to strain CNCM 1-1582, such as, for example, VOTiVO™, BioNem) or one of the following known active compounds: 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934), 4-{[(6-bromopyridin-3-yl)methyl](2-fluoroethyl)amino}furan-2(5H)-one (known from WO2007/115644), 4-{[(6-fluoropyridin-3-yl)methyl](2,2-difluoroethyl)amino}furan-2(5H)-one (known from WO2007/115644), 4-{[(2-chloro-1,3-thiazol-5-yl)methyl](2-fluoroethyl)amino}furan-2(5H)-one (known from WO2007/115644), 4-{[(6-chlorpyridin-3-yl)methyl](2-fluoroethyl)amino}furan-2(5H)-one (known from WO2007/115644), Flupyradifurone, 4-{[(6-chlor-5-fluoropyridin-3-yl)methyl](methyl)amino}furan-2(5H)-one (known from WO2007/115643), 4-{[(5,6-dichloropyridin-3-yl)methyl](2-fluoroethyl)amino}furan-2(5H)-one (known from WO2007/115646), 4-{[(6-chloro-5-fluoropyridin-3-yl)methyl](cyclopropyl)amino}furan-2(5H)-one (known from WO2007/115643), 4-{[(6-chloropyridin-3-yl)methyl](cyclopropyl)amino}furan-2(5H)-one (known from EP-A-0 539 588), 4-{[(6-chlorpyridin-3-yl)methyl](methyl)amino}furan-2(5H)-one (known from EP-A-0 539 588), {[1-(6-chloropyridin-3-yl)ethyl](methyl)oxido-λ4-sulfanylidene}cyanamide (known from WO2007/149134) and its diastereomers {[(1R)-1-(6-chloropyridin-3-yl)ethyl](methyl)oxido-λ4-sulfanylidene}cyanamide (A) and {[(1S)-1-(6-chloropyridin-3-yl)ethyl](methyl)oxido-×4-sulfanylidene}cyanamide (B) (also known from WO2007/149134) as well as Sulfoxaflor and its diastereomers [(R)-methyl(oxido){(1R)-1-[6-(trifluoromethyl)pyridin-3-yl]ethyl}-λ4-sulfanylidene]cyanamide (A1) and [(S)-methyl(oxido) {(1S)-1-[6-(trifluoromethyl)pyridin-3-yl]ethyl}-λ4-sulfanylidene]cyanamide (A2), referred to as group of diastereomers A (known from WO2010/074747, WO2010/074751), [(R)-methyl(oxido){(1S)-1-[6-(trifluoromethyl)pyridin-3-yl]ethyl}-λ4-sulfanylidene]cyanamide (B1) and [(S)-methyl(oxido){(1R)-1-[6-(trifluoromethyl)pyridin-3-yl]ethyl}-λ4-sulfanylidene]cyanamide (B2), referred to as group of diastereomers B (also known from WO2010/074747, WO2010/074751), and 11-(4-chloro-2,6-dimethylphenyl)-12-hydroxy-1,4-dioxa-9-azadispiro[4.2.4.2]tetradec-11-en-10-one (known from WO2006/089633), 3-(4′-fluoro-2,4-dimethylbiphenyl-3-yl)-4-hydroxy-8-oxa-1-azaspiro[4.5]dec-3-en-2-one (known from WO2008/067911), 1-{2-fluoro-4-methyl-5-[(2,2,2-trifluorethyl)sulfinyl]phenyl}-3-(trifluoromethyl)-1H-1,2,4-triazol-5-amine (known from WO2006/043635), [(3S,4aR,12R,12aS,12bS)-3-[(cyclopropylcarbonyl)oxy]-6,12-dihydroxy-4,12b-dimethyl-11-oxo-9-(pyridin-3-yl)-1,3,4,4a,5,6,6a,12,12a,12b-decahydro-2H,11H-benzo[f]pyrano[4,3-b]chromen-4-yl]methyl cyclopropanecarboxylate (known from WO2008/066153), 2-cyano-3-(difluoromethoxy)-N,N-dimethylbenzenesulfonamide (known from WO2006/056433), 2-cyano-3-(difluoromethoxy)-N-methylbenzenesulfonamide (known from WO2006/100288), 2-cyano-3-(difluoromethoxy)-N-ethylbenzenesulfonamide (known from WO2005/035486), 4-(difluoromethoxy)-N-ethyl-N-methyl-1,2-benzothiazol-3-amine 1,1-dioxide (known from WO2007/057407), N-[1-(2,3-dimethylphenyl)-2-(3,5-dimethylphenyl)ethyl]-4,5-dihydro-1,3-thiazol-2-amine (known from WO2008/104503), {1′-[(2E)-3-(4-chlorophenyl)prop-2-en-1-yl]-5-fluorospiro[indole-3,4′-piperidin]-1(2H)-yl}(2-chloropyridin-4-yl)methanone (known from WO2003/106457), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl ethyl carbonate (known from WO2009/049851), 4-(but-2-yn-1-yloxy)-6-(3,5-dimethylpiperidin-1-yl)-5-fluoropyrimidine (known from WO2004/099160), (2,2,3,3,4,4,5,5-octafluoropentyl)(3,3,3-trifluoropropyl)malononitrile (known from WO2005/063094), (2,2,3,3,4,4,5,5-octafluoropentyl)(3,3,4,4,4-pentafluorobutyl)malononitrile (known from WO2005/063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO2007/040280), Flometoquin, PF1364 (CAS-Reg. No. 1204776-60-2) (known from JP2010/018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO2007/075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO2007/075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005/085216), 4-{[(6-chloropyridin-3-yl)methyl](cyclopropyl)amino}-1,3-oxazol-2(5H)-one, 4-{[(6-chloropyridin-3-yl)methyl](2,2-difluoroethyl)amino}-1,3-oxazol-2(5H)-one, 4-{[(6-chloropyridin-3-yl)methyl](ethyl)amino}-1,3-oxazol-2(5H)-one, 4-{[(6-chloropyridin-3-yl)methyl](methyl)amino}-1,3-oxazol-2(5H)-one (all known from WO2010/005692), NNI-0711 (known from WO2002/096882), 1-acetyl-N-[4-(1,1,1,3,3,3-hexafluoro-2-methoxypropan-2-yl)-3-isobutylphenyl]-N-isobutyryl-3,5-dimethyl-1H-pyrazole-4-carboxamide (known from WO2002/096882), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-1,2-diethylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), (5RS,7RS;5RS,7SR)-1-(6-chloro-3-pyridylmethyl)-1,2,3,5,6,7-hexahydro-7-methyl-8-nitro-5-propoxyimidazo[1,2-a]pyridine (known from WO2007/101369), 2-{6-[2-(5-fluoropyridin-3-yl)-1,3-thiazol-5-yl]pyridin-2-yl}pyrimidine (known from WO2010/006713), 2-{6-[2-(pyridin-3-yl)-1,3-thiazol-5-yl]pyridin-2-yl}pyrimidine (known from WO2010/006713), 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide (known from WO2010/069502), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO2010/069502), N-[2-(tert-butylcarbamoyl)-4-cyano-6-methylphenyl]-1-(3-chloropyridin-2-yl)-3-{[5-(trifluoromethyl)-1H-tetrazol-1-yl]methyl}-1H-pyrazole-5-carboxamide (known from WO2010/069502), N-[2-(tert-butylcarbamoyl)-4-cyano-6-methylphenyl]-1-(3-chloropyridin-2-yl)-3-{[5-(trifluoromethyl)-2H-tetrazol-2-yl]methyl}-1H-pyrazole-5-carboxamide (known from WO2010/069502), (1E)-N-[(6-chloropyridin-3-yl)methyl]-N′-cyano-N-(2,2-difluoroethyl)ethanimidamide (known from WO2008/009360), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), and methyl 2-[3,5-dibromo-24 {[3-bromo-1-(3-chloropyridin-2-yl)-1H-pyrazol-5-yl]carbonyl}amino)benzoyl]-2-ethyl-1-methylhydrazinecarboxylate (known from WO2011/049233), and combinations thereof.

### Micronutrients and Micronutrient-Containing Compounds:

In context of the present invention micronutrients and micronutrient-containing compounds relates to compounds selected from the group consisting of active ingredients containing at least one metal ion selected from the group consisting of zinc, manganese, molybdenum, iron and copper or the micronutrient boron. More preferably these micronutrients and micronutrient-containing compounds are selected from the group consisting of the zinc containing compounds Propineb, Polyoxin Z (zinc salt), Zineb, Ziram, zinc thiodazole, zinc naphthenate and Mancozeb (also containing manganese), the manganese containing compounds Maneb, Metiram and Mancopper (also containing copper), the iron containing compound Ferbam, copper (Cu) and the copper containing compounds Bordeaux mixture, Burgundy mixture, Cheshunt mixture, copper oxychloride, copper sulphate, basic copper sulphate (e.g. tribasic copper sulphate), copper oxide, copper octanoate, copper hydroxide, oxine-copper, copper ammonium acetate, copper naphthenate, chelated copper (e.g. as amino acid chelates), mancopper, acypetacs-copper, copper acetate, basic copper carbonate, copper oleate, copper silicate, copper zinc chromate, cufraneb, cuprobam, saisentong, and thiodiazole-copper, and combinations thereof.

More preferably the micronutrients and micronutrient-containing compounds are selected from the group consisting of (4.1) copper (Cu), (4.2) copper-hydroxyde, (4.3) copper-sulphate, (4.4) copper-oxychloride, (4.5) Propineb and (4.6) Mancozeb. Even more preferably the micronutrients and micronutrient-containing compounds are selected from the group consisting of (4.2) copper-hydroxyde, (4.3) copper-sulphate, and (4.5) Propineb.

Lipochito-oligosaccharide compounds (LCO) (5).

A preferred combination partner from the group of fungicides is (2.1) fosetyl-Al (fosetyl-aluminium).

Further preferred combination partners from the group of fungicides are selected from strobilurins, fungicides belonging to the group of inhibitors of the respiratory chain at complex III, for example (3.1) ametoctradin, (3.2) amisulbrom, (3.3) azoxystrobin, (3.4) cyazofamid, (3.5) coumethoxystrobin, (3.6) coumoxystrobin, (3.7) dimoxystrobin, (3.8) enestroburin (WO 2004/058723), (3.9) famoxadone (WO 2004/058723), (3.10) fenamidone (WO 2004/058723), (3.11) fenoxystrobin, (3.12) fluoxastrobin (WO 2004/058723), (3.13) kresoxim-methyl (WO 2004/058723), (3.14) metominostrobin (WO 2004/058723), (3.15) orysastrobin (WO 2004/058723), (3.16) picoxystrobin (WO 2004/058723), (3.17) pyraclostrobin (WO 2004/058723), (3.18) pyrametostrobin (WO 2004/058723), (3.19) pyraoxystrobin (WO 2004/058723), (3.20) pyribencarb (WO 2004/058723), (3.21) triclopyricarb, (3.22) trifloxystrobin (WO 2004/058723), (3.23) (2E)-2-(2-{[6-(3-chloro-2-methylphenoxy)-5-fluoropyrimidin-4-yl]oxy}phenyl)-2-(methoxyimino)-N-methylethanamide (WO 2004/058723), (3.24) (2E)-2-(methoxyimino)-N-methyl-2-(2-{[({(1E)-1-[3-(trifluoromethyl)phenyl]ethylidene}amino)oxy]methyl}phenyl)ethanamide (WO 2004/058723), (3.25) (2E)-2-(methoxyimino)-N-methyl-2-{2-[(E)-({1-[3-(trifluoromethyl)phenyl]ethoxy}imino)methyl]phenyl}ethanamide (158169-73-4), (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide (326896-28-0), (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide (119899-14-8), (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulfanyl)methyl]phenyl}-3-methoxyprop-2-enoate, (3.31)N-(3-ethyl-3,5,5-trimethylcyclohexyl)-3-(formylamino)-2-hydroxybenzamide, (3.32) 2-{2-[(2,5-dimethylphenoxy)methyl]phenyl}-2-methoxy-N-methylacetamide and (3.33) (2R)-2-{2-[(2,5-dimethylphenoxy)methyl]phenyl}-2-methoxy-N-methylacetamide.

All named combination partners, as well as the dithiino-tetracarboximides of formula (I) of the present invention can, if their functional groups enable this, optionally form salts with suitable bases or acids.

Further, the dithiino-tetracarboximides of formula (I) of the present invention can be combined with at least one active compound selected from the group consisting of:

Acetic acid (e.g. naphthalene acetic acid), peracetic acid, organic acids (e.g. citric acid, lactic acid), amino acids (e.g. 1-arginine), humic acids, fulvic acids, boric acid, oxolinic acid, 1,2,3-Benzothiadiazole-7-thiocarboxylic acid-S-methyl-ester, 5-hydroxy-1,4-naphthalenedione, bromo-chloro-dimethylhydantoin, Trichloroisoyanuric acid, salicylic acid, dichlorophen, kanamycin, kasugamycin, streptomycin, strepromycin sulfate, oxytetracycline, gentamycin (e.g. gentamycin sulphate hydrate), imidacloprid, tebuconazole thiabendzole, thiram, teracep, octhilinone, quinoxyfen, azadirachtin, furanoflavone, forchlorfenuron, plant minerals (e.g. calcium, calcium calcium carbonate, hypochlorite, calcium EDTA), enzymes (e.g. protease, amylase, lipase), trace elements and chelated trace elements (e.g. as amino acid chelates), vitamins and plant extracts, salicylate derivatives, bioflavonoids and organic acids derived from vegetables and fruit, natural fruit extracted polyphenols, bitter orange oil, citrus extracts, chitosan, starch, seaweed extract, organosilicone, activated ionized silicon complex (Zumsil®), bee wax, urea, Bacillus subtilis, Bacillus amyloliquefaciens, Pseudomonas fluorescens, Pseudomonas putida, Pantoea agglomerans, Trichoderma koningii, Trichoderma harzianum, chlorine and chlorine compounds (e.g. chlorinated water, chlorine dioxide, sodium chlorite, sodium hypochlorite, hypochlorous acid, ammonium chloride, didecyl dimethyl ammonium chloride, benzalkonium chloride), oxygen, hydrogen peroxide (H₂O₂) and peroxygen compounds, hydrogen cyanamide, nickel (III) sulphate, sodium persulphate, phosphite, phosphate, Trisodium phosphate, phosphoric acid, inorganic nitrogen, silver and silver containing compounds (e.g. colloidal silver), glutaraldehyde, rhamnolipid (Zonix®).

In this context, the term “combination” or “formulation” means various combinations of at least two of the abovementioned additional active compounds which are possible, such as, for example, ready mixes, tank mixes (which is understood as meaning spray slurries prepared from the formulations of the individual active compounds by combining and diluting prior to the application) or combinations of these (for example, a binary ready mix of two of the abovementioned active compounds is made into a tank mix by using a formulation of the third individual substance). According to the invention, the individual active compounds may also be employed sequentially, i.e. one after the other, at a reasonable interval of a few hours or days, in the case of the treatment of seed for example also by applying a plurality of layers which contain different active compounds. Preferably, it is immaterial in which order the individual active compounds can be employed.

The dithiino-tetracarboximides of formula (I) can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, scattering, dusting, foaming, painting on and the like. It is furthermore possible to apply the compounds or formulations of the present invention by the ultra-low-volume method or to inject the active compound preparation, or the active compound itself, into the soil. The vegetative propagation material of the plants may also be treated.

The application rates may be varied within a substantial range, depending on the type of application. In the treatment of plant parts, the application rates of active compound are generally between 0.1 and 10 000 g/ha, preferably between 10 and 1000 g/ha. In the treatment of vegetative propagation material, the application rates of active compound are generally between 0.001 and 50 g per kilogram of vegetative propagation material, preferably between 0.01 and 10 g per kilogram of vegetative propagation material. In the treatment of the soil, the application rates of active compound are generally between 0.1 and 10 000 g/ha, preferably between 1 and 5000 g/ha.

The active compound formulations of the present invention comprise an effective and non-phytotoxic amount of the active ingredients with the expression “effective and non-phytotoxic amount” means an amount of the ingredients and the active compositions according to the invention which is sufficient for controlling or destroying pathogenic bacterial organisms present or liable to appear on the plants, by notably avoiding the development of resistant strains to the active ingredients and in each case does not entail any appreciable symptom of phytotoxicity for the said crops. Such an amount can vary within a wide range depending on the pathogen to be combated or controlled bacteria, the type of crop, the climatic conditions and the compounds included in the bactericide composition according to the invention. This amount can be determined by systematic field trials, which are within the capabilities of a person skilled in the art.

According to the present invention, a synergistic effect of e.g. fungicides is always present when the fungicidal activity of the active compound combinations exceeds the total of the activities of the active compounds when applied individually. The expected activity for a given combination of two active compounds (binary composition) can be calculated as follows:

$$E = x + y - \frac{x * y}{100}$$

in which E represents the expected percentage of inhibition of the disease for the combination of two fungicides at defined doses (for example equal to x and y respectively), x is the percentage of inhibition observed for the disease by the compound (A) at a defined dose (equal to x), y is the percentage of inhibition observed for the disease by the compound (B) at a defined dose (equal to y). When the percentage of inhibition observed for the combination is greater than E, there is a synergistic effect.

The expected activity for a given combination of three active compounds (ternary composition) can be calculated as follows:

$$E = X + Y + Z - ( \frac{X \cdot Y + X \cdot Z + Y \cdot Z}{100} ) + \frac{X \cdot Y \cdot Z}{10000}$$

wherein
X is the efficacy when active compound A is applied at an application rate of m ppm (or g/ha),
Y is the efficacy when active compound B is applied at an application rate of n ppm (or g/ha),
Z is the efficacy when active compound C is applied at an application rate of r ppm (or g/ha),
   E is the efficacy when the active compounds A, B and C are applied at application rates of m, n and r ppm (or g/ha), respectively.

The degree of efficacy, expressed in % is denoted. 0% means an efficacy which corresponds to that of the control while an efficacy of 100% means that no disease is observed.

If the actual activity exceeds the calculated value, then the activity of the combination is superadditive, i.e. a synergistic effect exists. In this case, the efficacy which was actually observed must be greater than the value for the expected efficacy (E) calculated from the abovementioned formula.

A further way of demonstrating a synergistic effect is the method of Tammes (cf. “Isoboles, a graphic representation of synergism in pesticides” in Neth. J. Plant Path., 1964, 70, 73-80).

### EXAMPLES

### Example 1

### Pseudomonas syringae Disease on Tomato

This example illustrates the efficacy of a composition according to the invention against Psuedomonas syringae disease on Tomato (Bacterial speck).

A standard experiment was conducted in Spain in 2011 to evaluate the performance of BCS-BB98685 against bacterial speck of tomato caused by the bacteria Pseudomonas syringae pv tomato.

Tomato plants were grown under plastic tunnel. Plots were artificially inoculated with a suspension of bacteria and treated with different experimental chemical formulations using a conventional sprayer. Four chemical sprays were applied within 7 days intervals. One artificial inoculation was performed one day after the third application.

Disease assessment was done on 3 tomato plants per plot, 11 days after the last application. Infected leaflets were sorted in 3 classes according to a severity scale (Class 1=1 spot/leaflet; Class 2=2-5 spots/leaflet; Class 3=>5 spots/leaflet). Then results were expressed as severity index and transformed in efficacy values using the Abbott formula.

Results from this experiment demonstrate that applications of a typical formulation containing 400 g of BCS-BB98685 per liter at the rate 500 g ai/ha can significantly reduce the level of bacterial infection on tomatoes, in comparison with untreated plots and standard treatment with copper oxychloride.

TABLE 1
Results from one trial in Spain, 2011: Pseudomonas syringae pv
tomato severity on tomato

| | | Disease | |
|---|---|---|---|
| | | severity % | efficacy |
| composition | Rate g ai/ha | index | (Abbott) |
| Untreated inoculated | — | 233 | — |
| Standard treatment (copper | 2450 | 101.3 | 56.5 |
| oxychloride) | | | |
| Compound I-1 400 SC | 500 | 58.0 | 75.1 |

### Example 2

### In Vitro-Test for the Calculation of the ED50-Value with Microorganisms

Wells of 96-hole microtitre plates are filled with 10 μl of a solution of the test compound in dimethyl sulfoxide (DMSO) and LB medium. Thereafter, into each well 190 μl of liquid LB medium is given that has been amended with an appropriate concentration of bacterial suspension.

With the aid of a photometer the extinction in all wells is measured at the wavelength of 620 nm. The microtiter plates are then transferred overnight onto a shaker at 28° C. and 85% relative humidity.

At the end of the incubation time the growth of the test organisms is measured again photometrically at the wavelength of 620 nm. The difference between the two extinction values (taken before and after incubation) is proportional to the growth of the test organism.

Based on the A extinction data from the different test concentrations and that of the untreated test organism (control) a dose-response curve is calculated. The concentration that is necessary to give 50% growth inhibition is defined and reported as ED50-value (=Effective Dose that causes 50% growth inhibition) in ppm (=mg/1).

TABLE 2

| active compound | microorganism | ED50- value |
|---|---|---|
| I-1 | Erwinia amylovora | 4.79 ppm |
| I-1 | Xanthomonas campestris | 31.22 ppm |

1. A dithiino-tetracarboximide of formula (I) 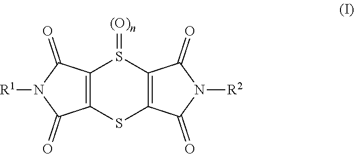
in which R1 and R2 are identical and represent methyl, ethyl, n-propyl or isopropyl, and n represents 0 or 1, or an agrochemically acceptable salt thereof,
capable of being used for controlling one or more bacterial harmful organisms in one or more useful plants, wherein the bacterial harmful organisms are selected from the group consisting of Acidovorax avenae, Burkholderia spec., Burkholderia glumae, Candidatus Liberibacter spec., Corynebacterium, Erwinia spec., Pseudomonas syringae, Pseudomonas syringae pv. actinidae, Pseudomonas syringae pv. glycinea, Pseudomonas syringae pv. tomato, Pseudomonas syringae pv. lachrymans, Pseudomonas tumefaciens (=Agrobacterium tumefaciens) Streptomyces spp., Xanthomonas spp., Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. viticola, Xanthomonas fragariae and Xanthomonas transluscens or Xylella fastidiosa.

2. A dithiino-tetracarboximide according to claim 1, selected from the group consisting of
(I-1) 2,6-dimethyl-1H,5H-[1,4]dithiino[2,3-c:5,6-c′]dipyrrole-1,3,5,7(2H,6H)-tetrone (i.e. R1=R2=methyl, n=0)
(I-2) 2,6-diethyl-1H,5H-[1,4]dithiino[2,3-c:5,6-c′]dipyrrole-1,3,5,7(2H,6H)-tetrone (i.e. R1=R2=ethyl, n=0)
(I-3) 2,6-dipropyl-1H,5H-[1,4]dithiino[2,3-c:5,6-c′]dipyrrole-1,3,5,7(2H,6H)-tetrone (i.e. R1=R2=propyl, n=0)
(I-4) 2,6-diisopropyl-1H,5H-[1,4]dithiino[2,3-c:5,6-c′]dipyrrole-1,3,5,7(2H,6H)-tetrone (i.e. R1=R2=isopropyl, n=0)
(I-5) 2,6-dimethyl-1H,5H-[1,4]dithiino[2,3-c:5,6-c′]dipyrrole-1,3,5,7(2H,6H)-tetrone 4-oxide (i.e. R1=R2=methyl, n=1).

3. A dithiino-tetracarboximide according to claim 1, wherein the the dithiino-tetracarboximide of formula (I) is (I-1) 2,6-dimethyl-1H,5H-[1,4]dithiino[2,3-c:5,6-c′]dipyrrole-1,3,5,7(2H,6H)-tetrone.

4. A dithiino-tetracarboximide according to claim 1, wherein the bacterial harmful organisms are selected from the group consisting of Acidovorax avenae, Burkholderia spec., Burkholderia glumae, Candidatus Liberibacter spec., Liberibacter asiaticus (Las), Corynebacterium, Erwinia amylovora, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia herbicola, Erwinia stewartiii, Erwinia uredovora, Dickeya dedantii, Dickeya solani, Pseudomonas syringae, Pseudomonas syringae pv. actinidae, Pseudomonas syringae pv. glycinea, Pseudomonas syringae pv. lachrymans, Pseudomonas syringae pv. tomato, Pseudomonas tumefaciens (=Agrobacterium tumefaciens), Streptomyces scabies, Xanthomonas ampelina, Xanthomonas axonopodis, Xanthomonas axonopodis pv. citri, Xanthomonas axonopodis pv. glycines, Xanthomonas campestris, Xanthomonas campestris pv. musacearum, Xanthomonas campestris pv. pruni, Xanthomonas campestris pv. viticola, Xanthomonas fragariae and Xanthomonas translucens or Xylella fastidiosa.

5. A dithiino-tetracarboximide according to claim 1, wherein the bacterial harmful organisms are selected from the group consisting of Burkholderia glumae, Candidatus Liberibacter spec., Liberibacter asiaticus (Las), Xanthomonas ampelina, Xanthomonas axonopodis pv. citri, Erwinia amylovora, Erwinia carotovora (=Pectobacterium carotovorum), Dickeya dedantii, Dickeya solani, Pseudomonas syringae, Pseudomonas syringae pv. actinidae, Pseudomonas syringae pv. glycinea, Pseudomonas syringae pv. lachrymans, Pseudomonas syringae pv. tomato, Pseudomonas tumefaciens (=Agrobacterium tumefaciens), Xanthomonas axonopodis pv. glycines, Xanthomonas campestris pv. pruni, Xanthomonas campestris pv. viticola and Xanthomonas campestris or Xylella fastidiosa.

6. A dithiino-tetracarboximide according to claim 1, wherein the useful plants are selected from the group consisting of fruit crops, vegetables, potatoes, cereals, corn, rice and soybeans.

7. A dithiino-tetracarboximide according to claim 1, wherein the useful plants are selected from the group consisting of grapewines, apples, bananas, citrus, kiwi, melons, peaches, pears, pineapple, pome fruit, pomegranate, cabbage, cauliflower, cucumbers, cucurbits, tomatoes, potatoes, wheat, rice and soybeans.

8. A dithiino-tetracarboximide according to claim 1, wherein the useful plants are selected from the group consisting of grapewines, apples, pears, citrus, kiwi, peaches, cucumbers, tomatoes, potatoes and wheat.

9. A dithiino-tetracarboximide according to claim 1 capable of being used for controlling Acidovorax avenae and/or Burkholderia glumae in rice, Candidatus Liberibacter spec. and/or Xanthomonas axonopodis pv. citri in citrus, Pseudomonas syringae pv. actinidae in Kiwi, Xanthomonas campestris and/or Xanthomonas campestris pv. pruni in peaches, Pseudomonas syringae pv. glycinea and/or Xanthomonas axonopodis pv. glycines in soybeans, Burkholderia spec. and/or Xanthomonas transluscens in cereals, Pseudomonas syringae, Pseudomonas syringae pv. tomato and/or Xanthomonas campestris in tomatoes, Pseudomonas syringae and/or Pseudomonas syringae pv. lachrymans in cucumbers, Erwinia atroseptica, Erwinia caratovora and/or Streptomyces scabies in potatoes; Xanthomonas ampelina, Xanthomonas campestris pv. viticola, Agrobacterium tumefaciens and Xylella fastidiosa in grapewines, Erwinia amylovora in apple and pears; Pectobacterium carotovorum in potatoes.

10. A dithiino-tetracarboximide according to claim 1 capable of being used for controlling Burkholderia glumae in rice, Candidatus Liberibacter spec. and/or Xanthomonas axonopodis pv. citri in citrus, Pseudomonas syringae pv. actinidae in Kiwi, Xanthomonas campestris and/or Xanthomonas campestris pv. pruni in peaches, Pseudomonas syringae pv. glycinea and/or Xanthomonas axonopodis pv. glycines in soybeans, Pseudomonas syringae and/or Pseudomonas syringae pv. tomato in tomatoes, Pseudomonas syringae and/or Pseudomonas syringae pv. lachrymans in cucumbers; Xanthomonas ampelina, Xanthomonas campestris pv. viticola, Agrobacterium tumefaciens and Xylella fastidiosa in grapewines; Erwinia amylovora in apple and pears; Pectobacterium carotovorum in potatoes.

11. A dithiino-tetracarboximide according to claim 1, present in a composition comprising at least one further compound selected from the group consisting of bactericides, antibiotics, fungicides, herbicides, micronutrients and micronutrient-containing compounds, and lipochito-oligosaccharide compounds (LCO).

12. A dithiino-tetracarboximide according to claim 11, wherein the at least one further compound is selected from the group consisting of fosetyl-Al, strobilurins, copper-containing compounds, propineb and mancozeb, lipochito-oligosaccharide compounds (LCO), kasugamycin, streptomycin, and oxytetracyclin.

13. A dithiino-tetracarboximide according to claim, wherein the wherein said composition comprises a host defense inducer that is isotianil and the at least one further compound is selected from the group consisting of fosetyl-Al, azoxystrobin, trifloxystrobin, copper-hydroxyde, copper-sulphate, copper-oxychloride, copper, propineb, mancozeb, lipochito-oligosaccharide compounds (LCO), kasugamycin, streptomycin, and oxytetracyclin.

14. Method for controlling bacterial harmful organisms in one or more useful plants, the method comprising treatment of one or more plants with a dithiino-tetracarboximide of formula (I) 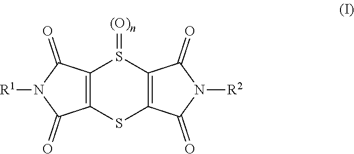
in which R1 and R2 are identical and represent methyl, ethyl, n-propyl or isopropyl, and n represents 0 or 1, or an agrochemically acceptable salt thereof.

15. Method according to claim 14, wherein the treated plants are transgenic plants.
